(19) United States
(12) Patent Application Publication
(10) Pub. No.: US 2018/0117148 A1
(43) Pub. Date: May 3, 2018

(54) COMPOUNDS, PHARMACEUTICAL COMPOSITIONS AND METHODS OF TREATMENTS OF IMMUNE RELATED DISEASES AND DISORDERS

(71) Applicant: Andrew J. Holman, Normandy Park, WA (US)

(72) Inventor: Andrew J. Holman, Normandy Park, WA (US)

(21) Appl. No.: 15/796,695

(22) Filed: Oct. 27, 2017

Related U.S. Application Data
(60) Provisional application No. 62/414,570, filed on Oct. 28, 2016.

(51) Int. Cl. A61K 39/395; A61P 37/00; A61P 37/02; A61K 31/5513; A61K 31/428; A61K 31/496; A61K 31/197; C07K 16/24

(52) U.S. Cl. CPC A61K 39/3955; A61P 37/00; A61P 37/02; A61K 31/5513; A61K 2039/505; A61K 31/496; A61K 31/197; C07K 16/241; A61K 31/428

(57) ABSTRACT

Provided herein are methods, pharmaceutical compositions and kits with combination therapies including biopharmaceuticals and neuroregulatory medicines for the treatment of immune related diseases and disorders.

### CROSS-REFERENCE TO RELATED APPLICATIONS

This application claims priority under 35 U.S.C. § 119(e) to U.S. Provisional Application No. 62/414,570 filed Oct. 28, 2016, which is hereby incorporated by reference in its entirety.

### FIELD

Compositions and methods in the field of medicine and chemistry are disclosed. Some of the disclosed embodiments are directed to medicinal compounds, medicinal compositions, as well as processes for their preparation and methods of their use. Some embodiments include methods of treatment of immune related diseases and disorders using the compounds and pharmaceutical compositions. In some embodiments, multiple compounds are combined in the compositions and treatment.

### BACKGROUND

The main purpose of medicinal therapeutics is identifying a specific, ideal therapeutic for each individual patient. In rheumatology, for example, after twenty years of use of biopharmaceuticals, none can yet be reliably targeted to those patients with particular diseases who are most likely to benefit. The current state of the art appears to be additionally flawed, since a minority of patients actually responds adequately to any currently available treatment.

Biologics have been used to treat a variety of diseases and disorders, including immune related diseases and disorders. One example of such a biopharmaceutical is etanercept which has been prescribed for immune related maladies. Although use of etanercept to treat inflammatory joint diseases like rheumatoid arthritis (RA) and psoriatic arthritis offers significant benefits, these diseases for many patients do not respond adequately. About 27% of patients achieve an American College of Rheumatology 70% improvement (ACR70) response and 20.5% receive a disease activity score (DAS) remission response.

As an inflammatory biomarker for autoimmune disease^{1,2}, the autonomic nervous system (ANS) has been assessed as a predictor of anti-TNF treatment outcome′ and as an adjunctive therapeutic target^{4,5,6} to elevate a stagnant remission rate in rheumatoid arthritis which has languished at 27% by ACR70 response⁷ for many years. The embodiments of the present disclosure relate to the concepts and issues discussed above. ¹Elenkov I J et al Pharmacological Reviews 2000; 52:595-638.²Koopman F A et al Mol Med 2011; 17(9-10):937-948.³Holman A J et al Autonomic Neurosci Basic Clinical 2008 Dec. 5; 143(1-2):58-67.⁴Koopman F A et al ACR 2012 abstract #451.⁵Shimizu M et al ACR 2003 abstract #114⁶Bencherif M et al Mol Life Sci. 2011; 68(6):931-949.⁷Moreland L W et al J Rheum 2006 May; 33(5):854-61.

(each of which is incorporated herein by reference)

### SUMMARY

Some embodiments relate to a method of treating an immune related disease or disorder comprising the co-administration of an effective amount of an immunomodulator and an effective amount of a neuroregulatory medicine to a patient in need of such treatment.

In some embodiments, the immunomodulator is a biopharmaceutical.

In some embodiments, the immunomodulator is at least one selected from the group consisting of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate.

In some embodiments, the immunomodulator is at least one selected from the group consisting of a JAK1 inhibitor, a JAK2 inhibitor, a JAK3 inhibitor, or a TYK2 inhibitor.

In some embodiments, the immunomodulator is at least one selected from the group consisting of etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, ruxolitinib, tofacitinib, oclacitinib, baricitinib, filgotinib, cucurbitacin gandotinib, lestaurtinib, momelotinib, pacritinib, pf-04965842, upadacitinib, peficitinib.

In some embodiments, the biopharmaceutical is etanercept.

In some embodiments, the neuroregulatory medicine is at least one selected from the group consisting of an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist.

In some embodiments, the neuroregulatory medicine is a benzodiazapene or a selective serotonin reuptake inhibitor.

In some embodiments, the neuroregulatory medicine is at least one selected from the group consisting of lorazepam, clonazepam, pramipexole, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3-Dichlorophenyl)-1-piperazinyl]cyclohexyl}-3-methoxybenzamide, (−)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin, Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone.

In some embodiments, the neuroregulatory medicine is at least one of lorazepam, clonazepam, pramipexole or trazodone.

In some embodiments, the co-administration is performed within a 72 hour period.

In some embodiments, the co-administration is performed within a 24 hour period.

In some embodiments, the subject is a mammal.

In some embodiments, the subject is human.

In some embodiments, the subject is human.

In some embodiments, the immune related disease or disorder is at least one selected from the group consisting of psoriatic arthritis, psoriasis, atopic dermatitis, atopic eczema, contact dermatitis, xerotic eczema, seborrheic dermatitis, neurodermitis, dyshidrosis, discoid eczema, venous eczema, dermatitis herpetiformis (Duhring's Disease), autoeczematization, dermatomyositis, hyper-IgE (Buckley) syndrome, Wiskott-Aldrich syndrome, anaphylaxis, food allergy, allergic reactions to venomous stings, acute urticarias, chronic urticarias, physical urticarias, aquagenic urticaria, cholinergic urticaria, cold urticaria (chronic cold urticaria), delayed pressure urticaria, dermatographic urticaria, heat urticaria, solar urticaria, vibration urticaria, adrenergic urticaria, urticaria angioedema, inflammatory bowel disease, Crohn's disease, ulcerative colitis, collagenous colitis, lymphocytic colitis, diversion colitis (diverticulitis), Behçet's syndrome, indeterminate colitis, celiac disease, irritable bowel syndrome, post-operative ileus, eosinophilic gastroenteropathy, gastritis, chronic allergic rhinitis, seasonal allergic rhinitis (hay-fever), allergic conjunctivitis, chemical conjunctivitis, neonatal conjunctivitis, Sjögren syndrome, open-angle glaucoma, dry eye disease, diabetic macular edema, mixed connective tissue disease, chronic obstructive pulmonary disease (COPD), allergic asthma, allergic bronchopulmonary aspergillosis, hypersensitivity pneumonitis, lung fibrosis, rheumatoid arthritis, juvenile rheumatoid arthritis, ankylosing spondylitis, spondyloarthropathy, systemic lupus erythematosus (SLE), subacute cutaneous lupus erythematosus, whipple, scleroderma, reactive arthritis, polymyalgia rheumatica, polymyositis, Dermatomyositis, Guillain-Barre syndrome, Hashimoto's thyroiditis, Grave's disease, temporal arteritis, liver disease, hepatitis, primary biliary cirrhosis, sclerosing cholangitis, autoimmune hepatitis, multiple sclerosis, and alopecia areata.

In some embodiments, the immune related disease or disorder is psoriatic arthritis or rheumatoid arthritis.

Some embodiments relate to pharmaceutical composition comprising: an effective amount of an immunomodulator, an effective amount of a neuroregulatory medicine and one or more pharmaceutically acceptable excipients.

Some embodiments relate to a kit comprising an effective amount of an immunomodulator, an effective amount of a neuroregulatory medicine and means for administration of one or both of the immunomodulator and the neuroregulatory medicine.

Some embodiments relate to a kit comprising an effective amount of an immunomodulator, an effective amount of a neuroregulatory medicine and administration tools such as a syringe or IV for one or both of the immunomodulator and the neuroregulatory medicine.

In some embodiments, the kit comprises instructions for co-administration of the biopharmaceutical and the neuroregulatory medicine.

The kit of claim 19, wherein the biopharmaceutical is one or more selected from the group consisting of etanercept, infliximab, adalimumab, tocilizumab, rituximab, ofatumumab, belimumab, epratuzumab, abatacept, golimumab, certolizumab pegol, sifalimumab, anakinra, canakinumab and rilonacept.

Some embodiments relate to a method of augmenting efficacy of an immunomodulator comprising co-administering an effective amount of a neuroregulatory medicine with the immunomodulator.

### BRIEF DESCRIPTION OF THE DRAWINGS

FIG. 1 is a graphical representation of mean joint count by disease.

FIG. 2 is a graphical representation of subjects who discontinued concomitant medications by disease.

### DETAILED DESCRIPTION

The present embodiments are directed to compositions and methods related to the treatment of immune related diseases and disorders. In some embodiments the compositions include an immunomodulator and in some embodiments, the compositions include small molecule compounds or others compounds. In some embodiments, the immunomodulator is a biopharmaceutical. In some embodiments, the immune related disease or disorder is RA or psoriatic arthritis. Etanercept is an example of a biopharmaceutical used to treat autoimmune diseases by interfering with tumor necrosis factor (TNF), acting as a TNF inhibitor. Etanercept has been used in the treatment of RA with limited success. A plethora of possible reasons may explain this variable response to etanercept, including gender, disease severity and duration, as well as serologic and genetic factors. Some embodiments relate to the autonomic nervous system (ANS) and its association with the driving of an autoimmune reaction to excess. Unlike many other proposed mechanisms, the ANS is modifiable as discussed below for use in a broader therapeutic approach.

The ANS plays an important role in the intensity of many disease processes, including inflammatory autoimmune disease. Some embodiments relate to the use of neuroregulatory medications, which are often able to modulate ANS state, in combination with biopharmaceuticals used to treat immune related diseases and disorders. Such synergistic combination results in the attenuated expression of the autoimmune disease itself resulting in superior efficacy of the biopharmaceutical and ultimate patient result.

In some embodiments, adjunctive neuroregulatory medications, many of which reduce ANS sympathetic tone, are co-administered with immunomodulators, such as etanercept, to patients exhibiting an incomplete therapeutic response to traditional immunosuppression (such as a biopharmaceutical combined with disease-modifying antirheumatic drugs (DMARDs), steroids (e.g. prednisone) and/or nonsteroidal anti-inflammatory drugs (NSAIDs)).

### Definitions

In accordance with the present disclosure and as used herein, the following terms are defined with the following meanings, unless explicitly stated otherwise. It is to be understood that both the foregoing general description and the following detailed description are exemplary and explanatory only and are not restrictive of the subject matter claimed. In this application, the use of the singular includes the plural unless specifically stated otherwise. In this application, the use of “or” means “and/or” unless stated otherwise. Furthermore, use of the term “including” as well as other forms, such as “includes,” and “included” is not limiting.

As used herein, “optional” or “optionally” means that the subsequently described event or circumstance does or does not occur, and that the description includes instances where the event or circumstance occurs and instances where it does not. For example, an optionally substituted group means that the group is unsubstituted or is substituted.

As used herein, the singular forms “a,” “an” and “the” include plural referents unless the context clearly dictates otherwise. Thus, for example, reference to a composition comprising “a therapeutic agent” includes compositions with one or a plurality of therapeutic agents.

The phrase “therapeutically effective amount” means an amount of a compound or a combination of compounds that partially or fully ameliorates, attenuates or eliminates one or more of the symptoms of a particular disease or condition or prevents, modifies, or delays the onset of one or more of the symptoms of a particular disease or condition. Such amount can be administered as a single dosage or can be administered according to a regimen, whereby it is effective. Repeated administration may be needed to achieve a desired result (e.g., treatment of the disease and/or condition).

The term “patient” refers to an animal being treated including a mammal, such as a dog, a cat, a cow, a horse, a sheep, monkey and a human. In some embodiments the patient is a mammal, either male or female. In some embodiments, the patient is a male or female human.

The term “sustained delivery” refers to an increase in the period in which there is a prolongation of therapeutically-effective drug levels due to the presence of the prodrug.

The terms “treating” or “treatment” of a disease includes inhibiting the disease (slowing or arresting or partially arresting its development), providing relief from the symptoms or side effects of the disease (including palliative treatment), and/or relieving the disease (causing regression of the disease).

The term “biopharmaceutical” refers to biological products such as proteins (including fusion proteins), vaccines, blood and blood components, allergenics, somatic cells, gene therapy components, tissues, and recombinant therapeutic proteins. Biologics can include sugars, proteins, or nucleic acids or complex combinations thereof, or may be living entities such as cells and tissues. Biologics can be isolated from a variety of natural sources such as human, animal, or microorganism and may be produced by a variety of methods including the use of recombinant DNA.

The term “immunomodulator” refers to a medicine used to help regulate or normalize the immune system by inducing, enhancing, suppressing or weakening an immune response in a patient.

The term “molecular pathway” refers to a series of molecular events in tissues such as a receptor modulating sequence or a biosynthesis sequence that is involved in physiological or pathophysiological functions of a living animal.

### Formulations

The disclosed compounds may be used alone or in combination with other treatments. These compounds, when used in combination with other agents, may be administered as a daily dose or an appropriate fraction of the daily dose (e.g., bid). The compounds may be administered after a course of treatment by another agent, during a course of therapy with another agent, administered as part of a therapeutic regimen, or may be administered prior to therapy by another agent in a treatment program.

Examples of pharmaceutically acceptable salts include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, palmoate, phosphate, polygalacturonate, stearate, succinate, sulfate, sulfosalicylate, tannate, tartrate, terphthalate, tosylate, and triethiodide.

Compositions containing the active ingredient may be in any form suitable for the intended method of administration. In some embodiments, the compounds of a method and/or composition described herein can be provided via oral administration, rectal administration, transmucosal administration, intestinal administration, enteral administration, topical administration, transdermal administration, intrathecal administration, intraventricular administration, intraperitoneal administration, intranasal administration, intraocular administration and/or parenteral administration.

When the compounds are administered via oral administration, for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient can be mixed with an inert solid diluent, for example calcium phosphate or kaolin, or as soft gelatin capsules wherein the active ingredient can be mixed with water or an oil medium, such as peanut oil, liquid paraffin or olive oil.

Formulations suitable for parenteral administration include aqueous and non-aqueous isotonic sterile injection solutions which may contain, for example, antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

In some embodiments unit dosage formulations contain a daily dose or unit, daily sub-dose, or an appropriate fraction thereof, of a drug. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

The above description discloses several methods and materials. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

The present embodiments include pharmaceutical formulations comprising one or more compounds described throughout in association with a pharmaceutically acceptable carrier. Preferably these formulations are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual, buccal, topical or rectal administration, or for administration by inhalation or insufflation. Also, the instant compounds can be administered to the body through Xenoport technology. XenoPort identifies and characterizes transporters throughout the body that are useful to drug delivery, then uses selected transporter proteins as “targets” and employs medicinal chemistry techniques to modify drugs into substrates for these transporters.

Alternatively, the formulations may be presented in a form suitable for once-daily, once-weekly or once-monthly administration; for example, an insoluble salt of the active compound may be adapted to provide a preparation for intramuscular injection. The pharmaceutical formulations described herein can be administered to a patient per se, or in pharmaceutical formulations where they are mixed with other active ingredients, as in combination therapy, or suitable pharmaceutically acceptable carriers or excipient(s). Techniques for formulation and administration of the compounds of the instant application may be found in “Remington's Pharmaceutical Sciences,” Mack Publishing Co., Easton, Pa., 18th edition, 1990.

For preparing solid pharmaceutical formulations such as tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation formulation containing a homogeneous mixture of a compound of the present embodiments, or a pharmaceutically acceptable salt thereof. When referring to these preformulation formulations as homogeneous, it is meant that the active ingredient is dispersed evenly throughout the formulation so that the formulation may be readily subdivided into equally effective unit dosage forms such as tablets, pills and capsules. This solid preformulation formulation is then subdivided into unit dosage forms of the type described above containing from about 0.01 to about 10,000 mg of the compounds of the present embodiments. Preferably the dosage is from about 50 to about 5000 mg; more preferably, the dosage is from about 450 to about 1800 mg; even more preferably, the dosage is from about 600 to about 1000 mg. The tablets or pills of the novel formulation can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Furthermore, compounds for the present embodiments can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the instant compounds can be formulated readily by combining the active compounds with pharmaceutically acceptable carriers well known in the art. Such pharmaceutically acceptable carriers enable the compounds of the present embodiments to be formulated as tablets, pills, dragees, capsules, liquids, gels, syrups, slurries, suspensions and the like, for oral ingestion by a patient to be treated. Pharmaceutical formulations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For buccal administration, the pharmaceutical formulations may take the form of tablets, lozenges, wafers and rapid-dissolve preparations formulated in conventional manner.

The compounds of the present embodiments can also be administered in the form of liposome pharmaceutical formulations, such as small unilamellar vesicles, large unilamellar vesicles and multilamellar vesicles. Liposomes can be formed from a variety of phospholipids, such as cholesterol, stearylamine or phosphatidylcholines.

Further disclosed herein are various pharmaceutical formulations well known in the pharmaceutical art for uses that include intraocular, intranasal, and intraauricular delivery. Suitable penetrants for these uses are generally known in the art. Pharmaceutical formulations for intraocular delivery include aqueous ophthalmic solutions of the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts.

Alternatively, other delivery systems for hydrophobic pharmaceutical compounds may be employed. Liposomes and emulsions are well known examples of delivery vehicles or pharmaceutical acceptable carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The dosage regimen utilizing the compounds of the present embodiments is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the pharmaceutical formulation's availability to target sites. This involves a consideration of the distribution, equilibrium, and elimination of the compounds. Advantageously, compounds of the present embodiments may be administered, for example, in a single daily dose, or the total daily dosage may be administered in divided doses of two, three or four times daily.

In the methods of the present present embodiments, the pharmaceutical formulations herein described in detail are typically administered in accordance with conventional pharmaceutical practices.

For instance, for oral administration in the form of a tablet or capsule, the compounds of the present embodiments can be combined with an oral, non-toxic pharmaceutically acceptable inert carrier such as ethanol, glycerol, water and the like. Moreover, when desired or necessary, suitable pharmaceutically acceptable carriers, such as, binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Some examples of pharmaceutically acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety.

The oral liquid formulations in which the present embodiments may be incorporated for administration orally include using pharmaceutically acceptable carriers, aqueous solutions, suitably flavoured syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous oral suspensions include synthetic and natural gums such as tragacanth, acacia, alginate, dextran, sodium carboxymethylcellulose, methylcellulose, polyvinyl-pyrrolidone or gelatin. Other dispersing agents which may be employed include glycerin and the like.

The daily dosage of the products may be varied over a wide range; e.g., from about 10 to about 10,000 mg per adult human per day. For oral administration, the formulations are preferably provided in the form of tablets containing about 0.001, 0.01, 0.1, 1, 10.0, 15.0, 25.0, 50.0, 100, 200, 300, 400, 500, 600, 700, 800, 900 1000, 2000, 3000, 4000, 5000, 6000, 7000, 8000, 9000 or 10,000 milligrams of the active ingredient for the symptomatic adjustment of the dosage to the patient to be treated. The instant pharmaceutical formulations typically contain from 10 mg to about 2000 mg of the instant compounds, preferably, from about 50 mg to about 1000 mg of active ingredient. An effective amount of the instant compounds is ordinarily supplied at a dosage level of from about 0.002 mg/kg to about 150 mg/kg of body weight per day. Preferably, the range is from about 0.02 to about 80 mg/kg of body weight per day, and especially from about 0.2 mg/kg to about 40 mg/kg of body weight per day. The compounds may be administered on a regimen of about 1 to about 10 times per day.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

Some embodiments relate to a kit which may include one or more compounds. In several embodiments, kits may further comprise suitable packaging and/or instructions for use of the compounds. Kits may also comprise a means for the delivery of the compound, such as an inhaler, spray dispenser (e.g., nasal spray), syringe for injection, needle, IV bag or pressure pack for capsules, tables, suppositories. The compounds can be in a dry or lyophilized form or in a solution, particularly a sterile solution. When the compounds are in a dry form, the kit may comprise a pharmaceutically acceptable diluent for preparing a liquid formulation. The kit may contain a device for administration or for dispensing the compositions, including, but not limited to, syringe, pipette, transdermal patch, or inhalant. Some embodiments relate to kits that contain sufficient dosages of the compounds or composition to provide effective treatment for an individual for an extended period, such as a week, 2 weeks, 3 weeks, 4 weeks, 6 weeks, or 8 weeks or more.

The following examples are provided for illustrative purposes only, and are in no way intended to limit the scope of the present embodiments.

### EXAMPLES

### Example 1

A retrospective, community-based study assessed consecutive patients initiating biologic treatment of inflammatory arthritis. Improved outcomes were measured by a 28-joint count (swollen and tender). Neuroregulatory medications, many of which reduce ANS sympathetic tone, were added to etanercept for patients exhibiting an incomplete therapeutic response to traditional immunosuppression (etanercept combined with DMARDs, prednisone and/or NSAID). Consecutive patients with either rheumatoid or psoriatic arthritis initiated etanercept (25 mg/BIW). For partial response, neuroregulatory medications were added. Neuroregulatory medication options included bedtime dosing of either lorazepam or clonazepam, pramipexole, trazodone, other antidepressants and anticonvulsants.

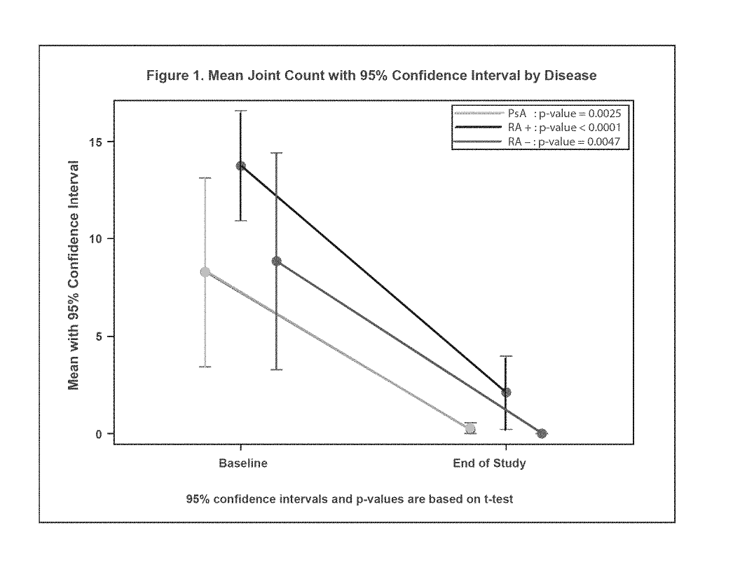

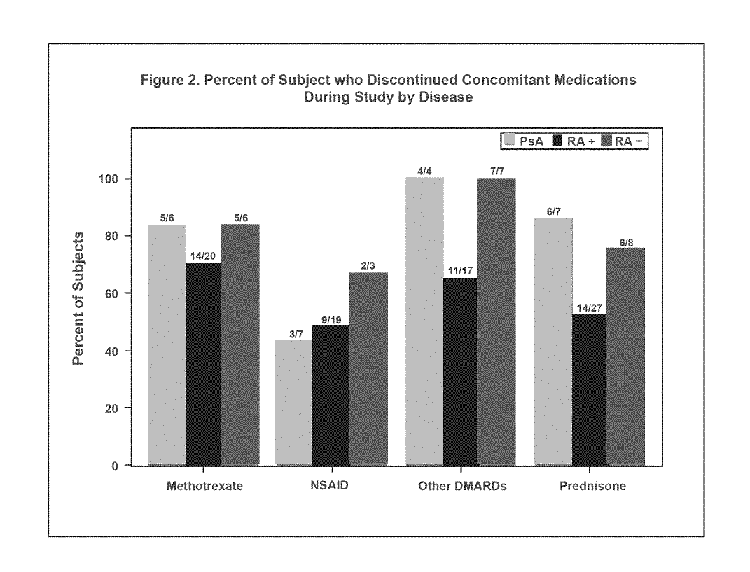

Primary outcome: “≥70% reduction in 28-joint count (tender and swollen). Statistical analysis: LOCF and logistic regression (see data in FIGS. 1 and 2).

Sixty-six patients (70% female, mean age of 50.8±13.0 years, mean duration of disease 9.7±6.4 years, mean prior DMARD use 2.3±1.4, mean ESR 26.6±27.8) initiated etanercept at 25 mg BIW for treatment of their inflammatory joint disease (39 RA+, 13 RA-, 14 PsA). All but five continued etanercept for a mean 20.7±7.7 months for an etanercept retention rate of 92%. Three subjects discontinued for personal reasons, one died of unrelated issues and one was lost to follow-up. Overall, initial joint count decreased after addition of etanercept from 11.6 to 1.3 with 79% achieving ≥70% reduction in joint count by LOCF (74% by BOCF). Baseline doses of prednisone and methotrexate were 6.7 mg/d (n=42) and 19.14 mg/wk (n=32), respectively. Ultimately, prednisone was discontinued in 62% (26 of 42); methotrexate was discontinued in 75% (24 of 32); other DMARDs were discontinued in 79% (22 of 28); and NSAIDs were discontinued in 48% (14 of 29). Neuroregulatory medications were used by 36 subjects (55%), including lorazepam 1-2 mg po qhs (18%), clonazepam 1-2 mg po qhs (15%), pramipexole 0.5-4.5 mg po qhs (29%), trazodone 50-100 mg po qhs (21%), other antidepressants (14%), and anti-epileptics (1%). Regression analysis identified the use of neuroregulatory medications as able to reconcile the clinical treatment response to etanercept seen in this cohort relative to historical etanercept treatment outcomes.

Patient Characteristics (at Initiation of Etanercept)

| | |
|---|---|
| Number of patients | 66 |
| Female | 70% |
| Diagnosis | |
| Rheumatoid arthritis (RF+) | 39 |
| Rheumatoid arthritis (RF−) | 13 |
| Psoriatic arthritis | 14 |
| Duration of disease (yrs) | 9.7 ± 6.4 |
| Mean 28-joint count | 11.6 ± 9.0 |
| Prior DMARDs | 2.3 ± 1.4 |
| ESR 26.6 | ±27.8 |
| Number of patients on prednisone | 42/66 |
| Prednisone (mean mg/d) | 6.7 |
| Number of patients on methotrexate | 32/66 |
| Methotrexate (mean mg/wk) | 19.14 |
| Number of patients on other DMARDs | 28/66 |
| Number of patients on NSAIDs | 29/66 |

Primary Efficacy Endpoint

79% (52/66) achieved “≥70% reduction in 28-joint count” (p<0.001). Mean 28-joint count decreased from 11.6±9.0 to 1.3±4.3. All but 5/66 patients continued etanercept for 20.7±7.7 months (92% retention). Three subjects discontinued for personal reasons unrelated to etanercept, one died of unrelated issued and one was lost to follow-up.

| | |
|---|---|
| Neuroregulatory medications | 36/66 (55%) |
| lorazepam (1-2 mg po qhs) | 12/66 (18%) |
| clonazepam (1-2 mg po qhs) | 10/66 (15%) |
| pramipexole (0.5-4.5 mg po qhs) | 19/66 (29%) |
| trazodone (50-100 mg po qhs) | 14/66 (21%) |
| other antidepressant | 9/66 (14%) |
| pregabalin (300 mg po qd) | 1/66 (2%) |

Adjunctive neuroregulatory medications combined with etanercept resulted in 79% of patients achieving the primary outcome of “≥70% reduction in 28-joint count.” (p<0.001) Beyond efficacy, 75% of patients discontinued methotrexate and 62% discontinued prednisone, indicating improvement in disease factors. The above data show the benefit of employing co-administering an adjunctive neuroregulatory ANS strategy to improve outcomes with immunosuppressive therapies including biopharmaceuticals.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15°, 10°, 5°, 3°, 1°, 0.1°, or otherwise. Similarly, in certain embodiments, the terms “generally perpendicular” and “substantially perpendicular” refer to a value, amount, or characteristic that departs from exactly perpendicular by less than or equal to 15°, 10°, 5°, 3°, 1°, 0.1°, or otherwise.

The above description discloses several methods and materials. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A method of treating an immune related disease or disorder comprising the co-administration of an effective amount of an immunomodulator and an effective amount of a neuroregulatory medicine to a patient in need of such treatment.

2. The method of claim 1, wherein the immunomodulator is a biopharmaceutical.

3. The method of claim 1, wherein the immunomodulator is at least one selected from the group consisting of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate.

4. The method of claim 1, wherein the immunomodulator is at least one selected from the group consisting of a JAK1 inhibitor, a JAK2 inhibitor, a JAK3 inhibitor, or a TYK2 inhibitor.

5. The method of claim 1, wherein the immunomodulator is at least one selected from the group consisting of etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, ruxolitinib, tofacitinib, oclacitinib, baricitinib, filgotinib, cucurbitacin gandotinib, lestaurtinib, momelotinib, pacritinib, pf-04965842, upadacitinib, peficitinib.

6. The method of claim 2, wherein the biopharmaceutical is etanercept.

7. The method of claim 1, wherein the neuroregulatory medicine is at least one selected from the group consisting of an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist.

8. The method of claim 1, wherein the neuroregulatory medicine is a benzodiazapene or a selective serotonin reuptake inhibitor.

9. The method of claim 1, wherein the neuroregulatory medicine is at least one selected from the group consisting of lorazepam, clonazepam, pramipexole, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3-Dichlorophenyl)-1-piperazinyl]cyclohexyl}-3-methoxybenzamide, (−)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin, Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone.

10. The method of claim 1, wherein the neuroregulatory medicine is at least one of lorazepam, clonazepam, pramipexole or trazodone.

11. The method of claim 1, further comprising the administration of another therapeutic to the patient.

12. The method of claim 1, wherein the co-administration is performed within a 72 hour period.

13. The method of claim 1, wherein the co-administration is performed within a 24 hour period.

14. The method of claim 1, wherein the immunomodulator the neuroregulatory medicine are administered to the patient within one week of each other.

15. The method of claim 1, wherein the immunomodulator the neuroregulatory medicine are administered to the patient within 72 hours of each other.

16. The method of claim 1, wherein the immunomodulator the neuroregulatory medicine are administered to the patient within 24 hours of each other.

17. The method of claim 1, wherein the subject is a mammal.

18. The method of claim 1, wherein the subject is human.

19. The method of claim 1, wherein the immune related disease or disorder is at least one selected from the group consisting of psoriatic arthritis, psoriasis, atopic dermatitis, atopic eczema, contact dermatitis, xerotic eczema, seborrheic dermatitis, neurodermitis, dyshidrosis, discoid eczema, venous eczema, dermatitis herpetiformis (Duhring's Disease), autoeczematization, dermatomyositis, hyper-IgE (Buckley) syndrome, Wiskott-Aldrich syndrome, anaphylaxis, food allergy, allergic reactions to venomous stings, acute urticarias, chronic urticarias, physical urticarias, aquagenic urticaria, cholinergic urticaria, cold urticaria (chronic cold urticaria), delayed pressure urticaria, dermatographic urticaria, heat urticaria, solar urticaria, vibration urticaria, adrenergic urticaria, urticaria angioedema, inflammatory bowel disease, Crohn's disease, ulcerative colitis, collagenous colitis, lymphocytic colitis, diversion colitis (diverticulitis), Behçet's syndrome, indeterminate colitis, celiac disease, irritable bowel syndrome, post-operative ileus, eosinophilic gastroenteropathy, gastritis, chronic allergic rhinitis, seasonal allergic rhinitis (hay-fever), allergic conjunctivitis, chemical conjunctivitis, neonatal conjunctivitis, Sjögren syndrome, open-angle glaucoma, dry eye disease, diabetic macular edema, mixed connective tissue disease, chronic obstructive pulmonary disease (COPD), allergic asthma, allergic bronchopulmonary aspergillosis, hypersensitivity pneumonitis, lung fibrosis, rheumatoid arthritis, juvenile rheumatoid arthritis, ankylosing spondylitis, spondyloarthropathy, systemic lupus erythematosus (SLE), subacute cutaneous lupus erythematosus, whipple, scleroderma, reactive arthritis, polymyalgia rheumatica, polymyositis, Dermatomyositis, Guillain-Barre syndrome, Hashimoto's thyroiditis, Grave's disease, temporal arteritis, liver disease, hepatitis, primary biliary cirrhosis, sclerosing cholangitis, autoimmune hepatitis, multiple sclerosis, and alopecia areata.

20. The method of claim 1, wherein the immune related disease or disorder is psoriatic arthritis or rheumatoid arthritis.

21. A pharmaceutical composition comprising: an effective amount of a immunomodulator, an effective amount of a neuroregulatory medicine and one or more pharmaceutically acceptable excipients.

22. The pharmaceutical composition of claim 21, wherein the immunomodulator is at least one selected from the group consisting of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate.

23. The pharmaceutical composition of claim 21 laur, wherein the neuroregulatory medicine is at least one selected from the group consisting of lorazepam, clonazepam, pramipexole, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3-Dichlorophenyl)-1-piperazinyl]cyclohexyl}-3-methoxybenzamide, (−)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin, Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone.
